(19) United States
(12) Patent Application Publication
(10) Pub. No.: US 2026/0345189 A1
(43) Pub. Date: Nov. 5, 2026

(54) BONE CUTTING DEVICE

(71) Applicant: Medmetalex Co. Ltd., Osaka (JP)

(72) Inventor: Amino Hirokazu, Osaka (JP)

(73) Assignee: Medmetalex Co. Ltd., Osaka (JP)

(21) Appl. No.: 19/555,359

(22) Filed: Mar. 3, 2026

(30) Foreign Application Priority Data
May 2, 2025 (JP) 2025-076756

(51) Int. Cl. A61B 17/32; A61F 2/46

(52) U.S. Cl. CPC A61B 17/32; A61F 2/46

(57) ABSTRACT

A cutting device for cutting bone even in the vicinity of an equator, and maintaining its cutting performance during surgery, includes: a hemispherical body having first cutting parts on its exterior, each including a part projecting outward, a first cutting blade formed by partially cutting the projecting part, and a through hole passing through the main body; a base rod at an edge of the body opposite its vertex; and a second cutting part at an end of the base rod, including a second surface obtained by cutting toward an inner side of the base rod from an end face of an end part thereof, a second cutting blade on a ridge line at a boundary between the second surface and the end face, and a concave surface, continuous with the second surface, obtained by cutting toward an inner side of the base rod.

### FIELD OF THE INVENTION

The present invention relates to a surgical bone cutting instrument, and particularly to a bone cutting instrument having a substantially hemispherical shell shape that forms a substantially hemispherical concave part in a bone.

### BACKGROUND OF THE INVENTION

In bone cutting instruments used for artificial hip joint surgery and the like, for example, as an instrument for embedding a hemispherical shell-shaped implant into an acetabulum on a pelvic side, there is a well-known instrument in which a large number of cutting blade parts for cutting bone are formed on an outer peripheral surface of a metallic main body having a substantially hemispherical shell shape, and such an instrument is used, in hip joint implant surgery, as a surgical instrument for a standard surgical procedure worldwide.

FIG. 5A shows a state in which a bone cutting instrument 110 is attached to a driver 120. A plurality of bone cutting parts 112 are formed on a main body 111 of the bone cutting instrument 110, and a bottom rod 116 is attached to a lower part thereof. A driver head 122 is disposed at a distal end of a shaft 121 of the driver 120, and an engagement recess 123 is formed in the driver head 122. The bottom rod 116 is engaged with the engagement recess 123, and the bone cutting instrument 110 rotates in accordance with rotation of the shaft 121 to cut bone by the bone cutting parts 112.

FIG. 5B is a diagram illustrating a state in which the driver 120 and the bone cutting instrument 110 assembled in FIG. 5A are applied to a pelvis 130. The pelvis 130 includes a hard cortical bone 131 on a surface thereof and a soft cancellous bone 132 inside thereof, and an acetabular fossa 133 into which a femoral head fits is formed in a dome shape using the bone cutting instrument 110.

FIGS. 6A, 6B and 6C show an instrument illustrated in FIG. 1 of Japanese patent JP 2005-504601 A. FIG. 6A shows a structure of an instrument 210, in which cutting parts 224 are formed on a wall 218 of a main body 212 that extends in a spherical shell shape from a vertex 216 on a rotation axis 214 to a visible edge part 222. As illustrated in FIG. 6C, a tongue-shaped blade 232 is formed on a cutting part 228 in the vicinity of the visible edge part 222. However, cutting is performed up to the visible edge part 222 after processing is performed up to a dotted-line portion in FIG. 6A, so that a hole 233 is cut away and opened on the visible edge part 222 side.

FIG. 7A shows an instrument illustrated in FIG. 4 of Japanese patent JP 2005-504601 A. A first cutting blade 330 of a first cutting part 324 and an opening part 325 are formed on a wall 318 of a main body 312. A base rod 334 is disposed at a visible edge part 322. A second blade 332 of a second cutting part 328 is formed on an end face of the base rod 334.

FIG. 7B shows an instrument illustrated in FIG. 5 of Japanese patent JP 2005-504601 A. A first blade 430 of a first cutting part 424 and an opening part 425 are formed on a wall 418 of a main body 412. A base rod 434 is disposed at a visible edge part 422. A second blade 432 (a knife-shaped blade) of a second cutting part 428 is formed on an end face of the base rod 434.

### SUMMARY OF THE INVENTION

In the bone cutting instrument 110 illustrated in FIGS. 5A and 5B, since the bone cutting part 112 is not provided in the vicinity of an equator, bone cutting in this portion is insufficient, and a diameter of an opening part of a dome-shaped hole formed in an acetabulum or the like becomes smaller than a planned size, which may cause difficulty when driving an acetabular implant.

In the instrument 210 illustrated in FIGS. 6A, 6B and 6C, the hole 233 is opened in the cutting blade part 228 in the vicinity of the visible edge part 222, and rigidity of the cutting blade part 228 is reduced, so that an event occurs in which the tongue-shaped blade 232 catches on bone during bone cutting because an end of the tongue-shaped blade 232 is chipped and the main body part is largely deformed, thereby causing a breakage accident, which has been a serious problem.

In the instrument illustrated in FIGS. 7A and 7B, bone in the vicinity of the visible edge parts 322 and 422 can be cut by using the second blades 332 and 432 disposed at the visible edge parts 322 and 422. However, cutting chips generated by the cutting are caught between surfaces of the main bodies 312 and 412 and the acetabular fossa, and the cutting chips stay on cutting edges of the second blades 332 and 432, so that the sharpness is quickly degraded.

The present invention has been made in view of the above circumstances, and an object thereof is to provide a bone cutting device, such as an acetabular reamer, that is capable of cutting bone reliably even in the vicinity of an equator, and that does not suffer a decrease in cutting performance during surgery.

A first aspect of the present invention is a bone cutting device including: a main body that extends in a substantially hemispherical shell shape from a vertex on a rotation axis. A plurality of first cutting parts are formed on an outer surface of the main body, Each of the first cutting parts includes a part projecting outward from the outer surface, a first cutting blade formed on a ridge line at a boundary between a first surface obtained by partially cutting the projecting part and a surface of the projecting part, and a through hole passing through the main body and being continuous to the first surface. A base rod is disposed at a peripheral edge part of the main body on a side opposite to the vertex, and a second cutting part is formed at an end part of the base rod, The second cutting part includes a second surface obtained by cutting toward an inner side of the base rod from an end face of the end part, a second cutting blade formed on a ridge line at a boundary between the second surface and the end face, and a concave surface obtained by cutting in a concave manner toward an inner side of the base rod while being continuous with the second surface, so that bone in the vicinity of the equator can be reliably cut away by the second cutting blade and cutting chips can be discharged from the concave surface, thereby preventing a decrease in sharpness during surgery.

In accordance with a second aspect of the present invention, in the bone cutting device according to the first aspect, the second cutting blade at the end part of the base rod is inscribed in a spherical surface in which the first cutting blades of the plurality of first cutting parts are inscribed.

Since the second cutting blade at the end part of the base rod is inscribed in the spherical surface in which the first cutting blades of the plurality of first cutting parts are inscribed, excessive cutting of bone in the vicinity of the equator does not occur even when the rotation axis deviates.

In accordance with a third aspect of the present invention, in the bone cutting device according to either of the first and second aspects, the base rod is engaged with an engagement recess formed in a driver head at a distal end of a driver for rotating the bone cutting device around the rotation axis.

In the third aspect of the present invention, since the base rod is engaged with the engagement recess formed in the driver head at the distal end of the driver for rotating the bone cutting device around the rotation axis, the base rod can be used for two purposes, that is, for forming the second cutting part, and for coupling with the driver, thereby making it possible to achieve cost reduction.

According to the present invention, it is possible to provide a bone cutting device, such as an acetabular reamer, that is capable of cutting bone reliably even in the vicinity of an equator, and that does not suffer a decrease in cutting performance during surgery.

### BRIEF DESCRIPTION OF THE DRAWING

FIG. 1A is a perspective view of a bone cutting device according to a first embodiment of the present invention;

FIGS. 1B and 1C are perspective views showing details of components of the device illustrated in FIG. 1A.

FIG. 2A is a perspective view of a bone cutting device according to a second embodiment of the present invention;

FIGS. 2B and 2C are perspective views showing details of components of the device illustrated in FIG. 2A.

FIG. 3 is a partial cross-sectional view of the bone cutting device according to the first embodiment of the present invention;

FIG. 4 is a partial cross-sectional view of a bone cutting device according to a third embodiment of the present invention;

FIG. 5A shows a bone cutting instrument and a driver in the related art, which are applied to a pelvis;

FIG. 5B is a view, partly in cross-section, illustrating the operation of the bone-cutting device of FIG. 5A.

FIG. 6A shows an instrument illustrated in FIG. 1 of Japanese patent JP 2005-504601 A;

FIG. 6B is a partial elevational view of the right side of the instrument in FIG. 6A;

FIG. 6C is an elevational view showing details of the blade in FIGS. 6A and 6B.

FIGS. 7A and 7B are perspective views of an instrument illustrated in FIGS. 4 and 5 of Japanese patent JP 2005-504601A;

FIGS. 8A, 8B and 8C illustrate the inclination of the rotation axis of an instrument having a cylindrical skirt part extending downward from an equator; and

FIG. 8D illustrates the enlargement of the diameter of the opening part of the instrument in FIGS. 8A, 8B and 8C when the axis of rotation is inclined;

FIG. 9 shows an instrument illustrated in FIG. 6 of Japanese patent JP 2005-504601 A; and

FIG. 10 shows an overhanging shape of a projecting part of the instrument in FIG. 9.

### DETAILED DESCRIPTION

The following describes embodiments of the present invention in detail based on the drawings. It should be noted that the following embodiments are essentially preferred examples, and are not intended to limit the scope of the present invention, an application thereof, or a use thereof.

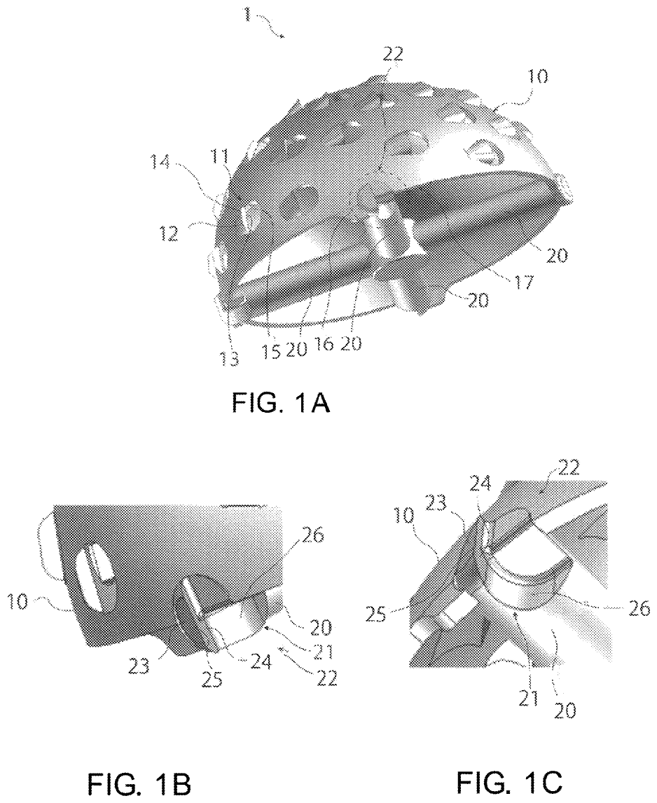

FIG. 1A shows a bone cutting device 1 according to a first embodiment of the present invention. A large number of first cutting parts 11 are formed on a surface of a main body 10. For clarity of the drawing, first cutting parts on the opposite side are not illustrated. In the first cutting part 11, a first surface 13 obtained by cutting a projecting part 12 is formed, and a ridge line at a boundary between a surface of the projecting part 12 and the first surface 13 serves as a first cutting blade 14. A through hole 15, passing through the main body 10, is formed to be continuous to the first surface 13. Cutting chips that have been cut away flow into an interior of the main body 10 through the through hole 15 and are discharged to the outside.

A base rod 20 is disposed at a peripheral edge part 17 of the main body 10. A second cutting part 22 (circled by a dotted line in FIG. 1A) is formed at an end part 21 of the base rod 20 (refer to FIG. 1B). A support part 16 extending downward from the main body 10 is formed, and this part serves to support the base rod 20. The base rod 20 and the main body 10 can be fixed to each other by welding, for example.

FIG. 1B is a view of FIG. 1A as seen obliquely from above. A second surface 24 is formed from an end face 23, and a ridge line at a boundary between a surface of the end face 23 and the second surface 24 serves as a second cutting blade 25. A concave surface 26 is formed from approximately a lower half of the second surface 24. Due to the presence of the concave surface 26, cutting chips are conveyed to an interior of the main body 10 and are discharged to the outside. Due to this discharge, cutting chips do not accumulate around the second cutting blade 25, so that the sharpness is not decreased during surgery. FIG. FIG. 1C is a view of FIG. 1A as seen obliquely from below.

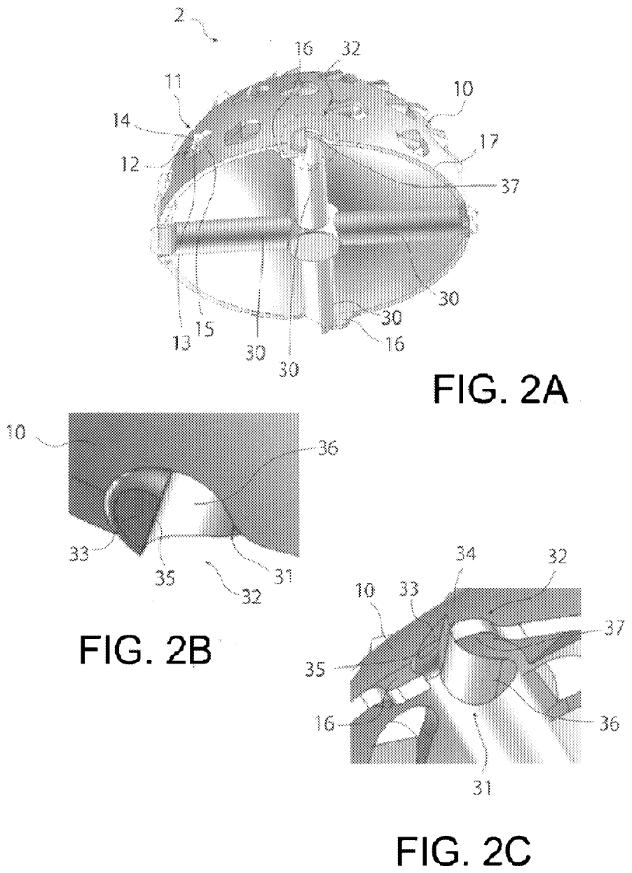

FIG. 2A shows a bone cutting device 2 according to a second embodiment of the present invention. The structure related to the main body 10 is the same as that in FIG. 1A, so that the description thereof will not be repeated.

A base rod 30 is disposed at a peripheral edge part 17 of the main body 10. A second cutting part 32 (circled by a dotted line in FIG. 1A) is formed at an end part 31 of the base rod 30 (refer to FIG. 2B). FIG. 2B is a view of FIG. 2A as seen obliquely from above. A second surface 34 is formed from an end face 33, and a ridge line at a boundary between a surface of the end face 33 and the second surface 34 serves as a second cutting blade 35. The second surface 34 is formed over an entire region of the end face 33, and a concave surface 36 is also formed over the entire region of the end face 33. Due to the presence of the concave surface 36, cutting chips are conveyed to an interior of the main body 10 and are discharged to the outside. Due to this discharge, cutting chips do not accumulate around the second cutting blade 35, so that the sharpness is not decreased during surgery. A gap 37 is provided between the concave surface 36 and the main body 10, so that chip discharge capability is improved. FIG. 2C is a view of FIG. 2A as seen obliquely from below.

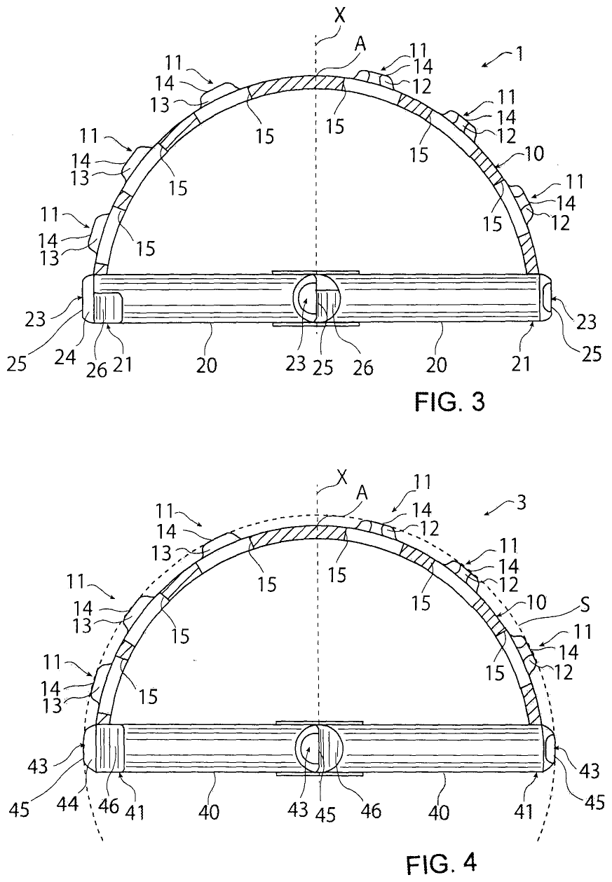

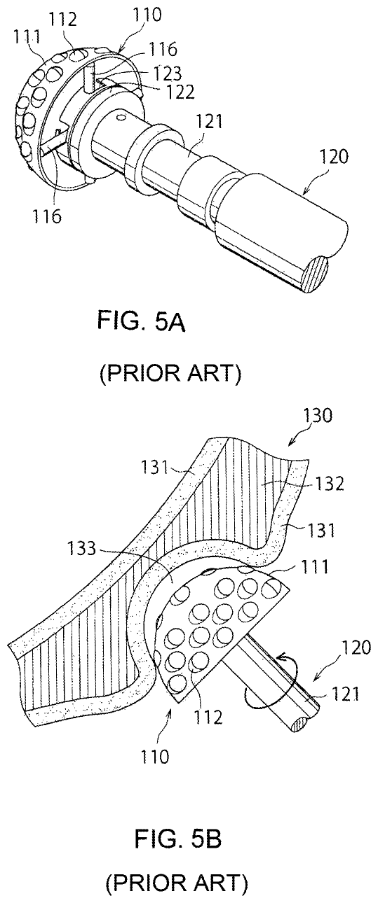

FIG. 3 is a partial cross-sectional view of the bone cutting device according to the first embodiment of the present invention illustrated in FIG. 1. The bone cutting device 1 includes the main body 10 that extends from a vertex A in a substantially hemispherical shape. The bone cutting device 1 is rotated around a rotation axis X by an electric driver (not illustrated) to cut cortical bone of an acetabular fossa by the first cutting blade 14 and the second cutting blade 25. The driver is like the driver 120 illustrated in FIG. 5.

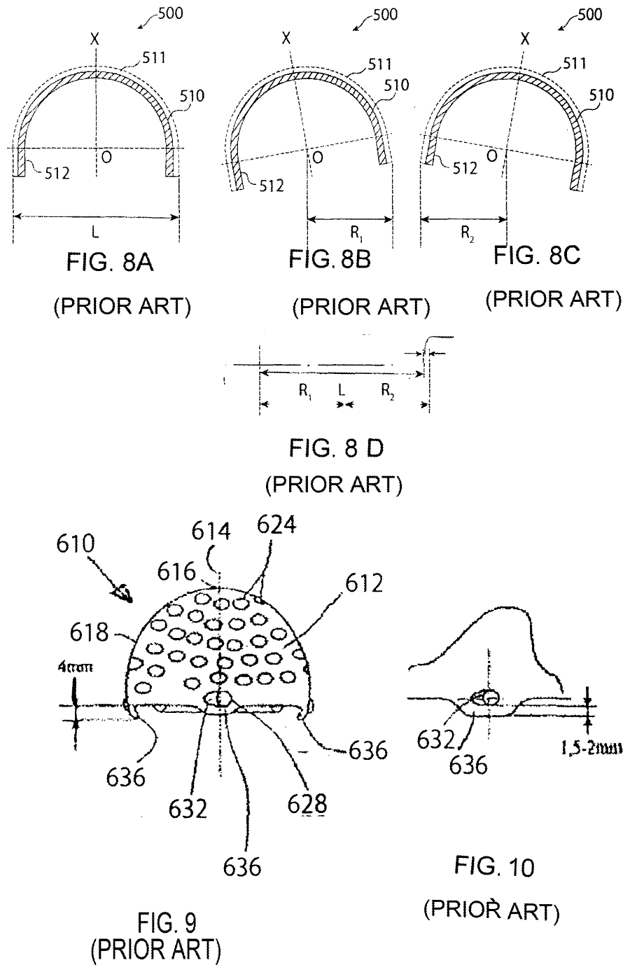

In a case of a bone cutting device 500 including a main body 510 and a cutting blade part 511 as illustrated in FIGS. 8A-8C, when cutting is performed vertically upward about the rotation axis X, a diameter of an opening part correctly becomes L, as shown in FIG. 8A. When cutting is performed in a state in which the rotation axis X is slightly inclined and deviates, as illustrated in 8B and 8C, the diameter of the opening part becomes (R₁+R₂), which is larger than the diameter L as illustrated in FIG. 8D, thereby possibly making it impossible to appropriately fix an acetabular implant even when it is driven in. FIG. 4 shows a bone cutting device according to the embodiment for solving the problem as described above with reference to FIGS. 8A-8D. FIG. 4 is a partial cross-sectional view of the bone cutting device according to a third embodiment of the present invention. Structures that are the same as those in FIG. 3 will not be described. An end face 43 at an end part 41 of a base rod 40 is different from the end face 23 illustrated in FIG. 3. The difference is that the end face 43 is formed in a curved manner so as to be inscribed in a spherical surface S in which the first cutting blade 14 of the first cutting part 11 is inscribed. A second surface 44, a second cutting blade 45, and a concave surface 46 are formed.

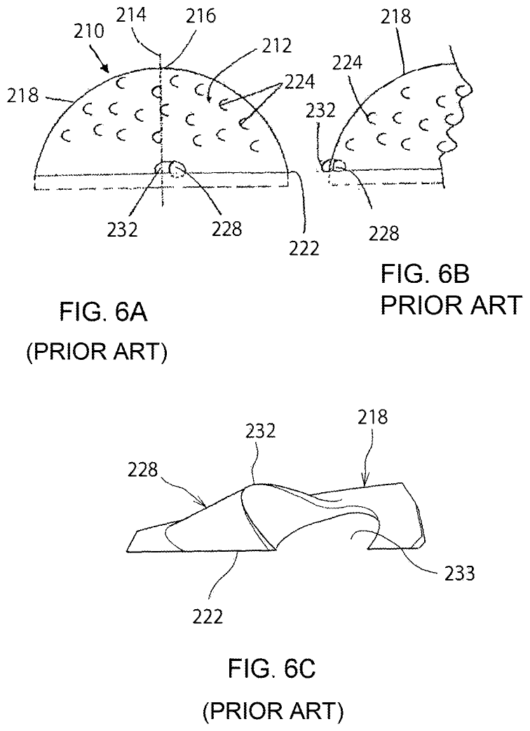

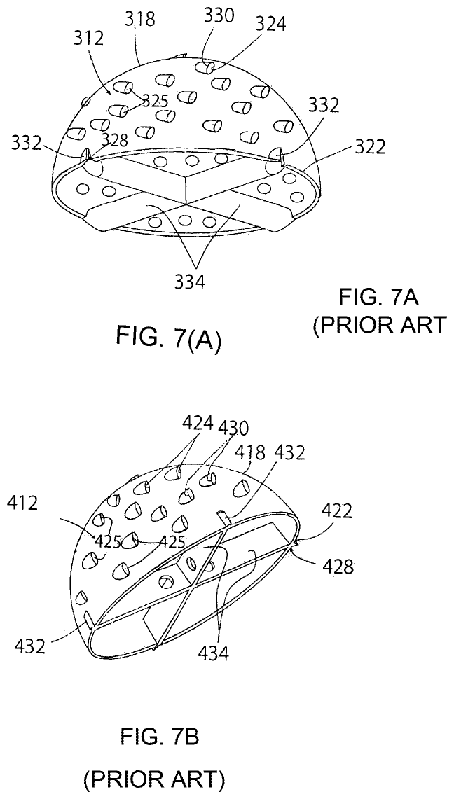

FIGS. 9 and 10 show an instrument illustrated in FIG. 6 of Japanese Patent JP 2005-504601 A. An instrument 610 spreads from a vertex 616 on a rotation axis 614, and a projecting part 636 overhangs beyond an equator. A first cutting part 624 is formed on a wall 618 of a main body 612. A second cutting part 628 including a second blade 632 is formed on the projecting part 636, and bone in the vicinity of the equator can be cut by the second blade 632. The reason the projecting part 636 has an overhanging shape is to prevent excessive cutting of bone in the vicinity of the equator when the orientation of the rotation axis 614 deviates. To form the projecting part 636 in an overhanging manner, a large number of process steps is required in a case of cutting processing, and even in a case of drawing processing, a great deal of effort is required. In a case of the bone cutting device illustrated in FIG. 4, the number of process steps as required for the instrument 610 illustrated in FIG. 9 is not required, and cost reduction can be achieved.

On the other hand, as in the third embodiment illustrated in FIG. 4, merely by forming the end face 43 into a shape inscribed in the spherical surface S, similarly to the instrument 610 illustrated in FIG. 9, it is possible to prevent excessive cutting of bone in the vicinity of the equator when the rotation axis X deviates.

### INDUSTRIAL APPLICABILITY

As described above, according to the present invention, it is possible to provide a bone cutting device, such as an acetabular reamer, that is capable of reliably cutting bone even in the vicinity of an equator and that does not suffer a decrease in cutting performance during surgery.

### REFERENCE SIGNS LIST

1; Bone cutting device
2; Bone cutting device
3; Bone cutting device
10; Main body
11; First cutting part
12; Projecting part
13; First surface
14; First cutting blade
15; Through hole
16; Support part
17; Peripheral edge part
20; Base rod
21; End part
22; Second cutting part
23; End face
24; Second surface
25; Second cutting blade
26; Concave surface
30; Base rod
31; End part
32; Second cutting part
33; End face
34; Second surface
35; Second cutting blade
36; Concave surface
37; Gap
40; Base rod
41; End part
43; End face
44; Second surface
45; Second cutting blade
46; Concave surface
X; Rotation axis
A; Vertex

What is claimed is:

1. A bone cutting device comprising:
a main body that extends in a substantially hemispherical shell shape from a vertex on a rotation axis;
a plurality of first cutting parts formed on an outer surface of the main body, the first cutting parts each including a projecting part projecting outward from the outer surface, a first cutting blade formed on a ridge line at a boundary between a first surface obtained by partially cutting the projecting part and a surface of the projecting part, and a through hole passing through the main body and being continuous to the first surface;
a base rod disposed at a peripheral edge part of the main body on a side opposite to the vertex; and
a second cutting part formed at an end part of the base rod, the second cutting part including a second surface obtained by cutting toward an inner side of the base rod from an end face of the end part, a second cutting blade formed on a ridge line at a boundary between the second surface and the end face, and a concave surface obtained by cutting in a concave manner toward an inner side of the base rod while being continuous to the second surface.

2. The bone cutting device according to claim 1, wherein the base rod is engaged with an engagement recess formed in a driver head at a distal end of a driver for rotating the bone cutting device around the rotation axis.

3. The bone cutting device according to claim 1, wherein the second cutting blade at the end part of the base rod is inscribed in a spherical surface in which the first cutting blades of the plurality of first cutting parts are inscribed.

4. The bone cutting device according to claim 3, wherein the base rod is engaged with an engagement recess formed in a driver head at a distal end of a driver for rotating the bone cutting device around the rotation axis.
